(19) United States
(12) Patent Application Publication
(10) Pub. No.: US 2026/0144432 A1
(43) Pub. Date: May 28, 2026

(54) ARTICULATION ASSEMBLIES FOR MEDICAL DEVICES

(71) Applicant: Boston Scientific Scimed, Inc., Maple Grove, MN (US)

(72) Inventor: Sean POWELL, Holden, MA (US)

(73) Assignee: Boston Scientific Scimed, Inc., Maple Grove, MN (US)

(21) Appl. No.: 19/398,369

(22) Filed: Nov. 24, 2025

Related U.S. Application Data
(60) Provisional application No. 63/725,618, filed on Nov. 27, 2024.

(51) Int. Cl. A61B 1/008; A61B 1/005

(52) U.S. Cl. CPC A61B 1/008; A61B 1/0055

(57) ABSTRACT

An articulation assembly for a medical device may include a plurality of discs and a plurality of actuation elements. Each actuation element may include a primary stem and a plurality of arms extending radially outward from the primary stem. Each arm of the plurality of arms may extend at least partially through at least two adjacent discs of the plurality of discs.

### CROSS-REFERENCE TO RELATED APPLICATION(S)

This application claims the benefit of priority from U.S. Provisional Application No. 63/725,618, filed on Nov. 27, 2024, which is incorporated by reference herein in its entirety.

### TECHNICAL FIELD

The present disclosure relates generally to endoscopic medical devices and assemblies. More particularly, in some embodiments, the disclosure relates to endoscopes configured for accessing target sites having space constraints, using, e.g., a flexible steerable shaft such as an articulation joint assembly disposed at a distal end of the endoscope.

### BACKGROUND

Endoscope devices generally include a flexible shaft, a working distal tip, and a flexible steerable shaft joining the working tip and the flexible shaft. The flexible steerable shaft may include a bendable articulation joint. The articulation joint may be bendable in limited numbers of degrees of freedom. It is desirable for an articulation joint configured to provide additional degrees of freedom. The present disclosure may solve one or more of these problems or other problems in the art. The scope of the disclosure, however, is defined by the attached claims and not the ability to solve a specific problem.

### SUMMARY OF THE DISCLOSURE

According to an example, an articulation assembly for a medical device may include a plurality of discs and a plurality of actuation elements. Each actuation element may include a primary stem and a plurality of arms extending radially outward from the primary stem. Each arm of the plurality of arms may extend at least partially through at least two adjacent discs of the plurality of discs.

Any of the devices disclosed herein may include any of the following features, alone or in any combination. Each disc of the plurality of discs may be equally spaced from one another in a straight configuration. Each arm of the plurality of arms may be configured to extend distally through a respective channel of a first disc of the at least two adjacent discs. Each respective channel may have a non-zero angle with respect to a central longitudinal axis of the articulation assembly. A distal end of each arm may include a termination feature. The termination feature may be secured within a second, distal disc of the at least two adjacent discs.

The articulation assembly may be configured to bend in at least four directions. In a first configuration of the articulation assembly, the articulation assembly may have a first length. In a second configuration, the articulation assembly may have a second length. The second length may be greater than the first length. In a third configuration, the articulation assembly has a third length. The third length may be less than the first length and the second length.

The plurality of discs may be configured to slide laterally relative to one another. Each arm of the plurality of arms may be coupled to the primary stem at a fork. The primary stem of each actuation member may extend at least partially through a respective Bowden sheath. The Bowden sheath may be a primary Bowden sheath. A portion of each arm of the plurality of arms may extend through a secondary Bowden sheath. The secondary Bowden sheath may extend between the joint and a proximal face of a first, proximal disc of the at least two adjacent discs.

Each disc of the plurality of discs may include a body defining a central opening. The primary stem of each actuation elements of the plurality of actuation elements may extend through the central opening. The plurality of discs may include at least six discs. The plurality of actuation elements may include at least six actuation elements. The articulation assembly may be configured to twist in at least one direction about a central longitudinal axis of the articulation assembly.

According to another example, an articulation assembly for a medical device may include a plurality of discs and a plurality of actuation elements arranged in pairs. Each disc may include a body defining a central opening. Each actuation element may include a primary stem and a plurality of arms extending radially outward from the primary stem. A distal end of each arm may be fixedly coupled within the body of a disc of the plurality of discs.

Any of the devices disclosed herein may include any of the following features, alone or in any combination. Each arm of the plurality of arms may be configured to extend distally through a respective channel of the body of at least one disc of the plurality of discs. The respective channel may have a non-zero angle with respect to a central longitudinal axis of the articulation assembly.

Each disc of the plurality of discs may be configured to slide laterally relative to one another in at least one configuration.

According to another example, an articulation joint for a medical device may include at least six discs and at least six actuation elements. Each disc of the at least six discs may include a body defining a central opening. Each actuation element may include a primary stem and a plurality of arms extending radially outward from the primary stem. A distal end of each arm of the plurality of arms may be fixedly coupled within each of the at least six discs.

Any of the devices disclosed herein may include the following feature, alone or in combination. Each arm of the plurality of arms may be configured to extend distally through a respective channel of each disc of the at least six discs.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary medical device, according to aspects of this disclosure.

FIG. 2 depicts an articulation joint assembly and distal tip of a medical device, according to aspects of this disclosure.

FIGS. 3A and 3B each depict different portions of the articulation joint assembly, according to aspects of this disclosure.

FIG. 4 depicts a cross-sectional view of a portion of the articulation joint assembly of FIG. 2, according to aspects of this disclosure.

FIGS. 5A-9D each depict a different configuration of the articulation joint assembly and distal tip of FIG. 2, according to aspects of this disclosure.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. In some drawings, arrows labeled “P” and “D” indicate proximal and distal directions, respectively. The term “exemplary” is used in the sense of “example,” rather than “ideal.” The term “approximately,” or like terms (e.g., “substantially”), includes values +/−10% of a stated value.

Drawbacks of conventional endoscopes include, for example, the inability to position or orient a distal tip in a desired position. For example, conventional articulation joints of endoscopes may have only two degrees of freedom, thus enabling a user to deflect, or articulate, the distal tip of the endoscope in up to four directions (e.g., up, down, left, or right). To extend, retract, or rotate the distal tip, the user may need to manipulate the entire endoscope. In other aspects, the user may need to remove and reinsert the endoscope into the subject, prolonging procedural times. Furthermore, these drawbacks can prevent the physician from properly visualizing and/or accessing areas of the body during procedures.

The disclosed assemblies provide up to four additional degrees of freedom of movement. For example, the disclosed assemblies may allow a distal tip of an endoscope to slide/translate left or right or to slide/translate forward or backward (i.e., up or down). The disclosed assemblies may also allow the distal tip of the endoscope to extend or retract. Furthermore, the disclosed assemblies may allow a shaft of the endoscope and the distal tip of the endoscope to rotate clockwise or counterclockwise.

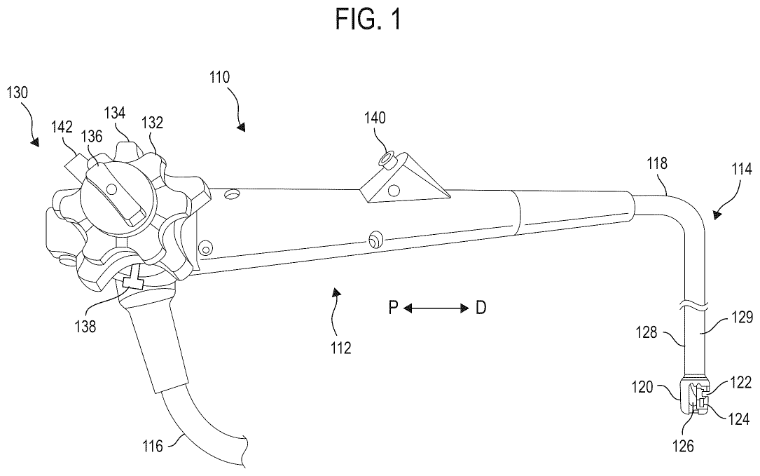

FIG. 1 depicts an exemplary medical device 110 having a handle 112 and an insertion portion 114. Medical device 100 may also include an umbilicus 116 for purposes of connecting medical device 110 to sources of, for example, air, water, suction, power, etc., as well as to image processing and/or viewing equipment. Although duodenoscopes and endoscopes (and combination devices that perform functions of duodenoscopes and endoscopes) are particularly referenced herein, the disclosure also encompasses other types of devices, such as bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to an endoscope herein should be understood to encompass any of the above medical devices.

Insertion portion 114 may include a sheath or shaft 118 and a distal tip 120. Distal tip 120 may include an imaging device 122 (e.g., a camera) and a lighting element 124 (e.g., a light emitting diode (LED) or an optical fiber). Although the term “lighting element” is used herein, it will be appreciated that the term “lighting element” may include a plurality of lighting elements (e.g., a plurality of LEDs or optical fibers). Distal tip 120 may be side facing. That is, imaging device 122 and lighting element 124 may face radially outward, perpendicularly, approximately perpendicularly, or otherwise transverse to a longitudinal axis of shaft 118 and distal tip 120 (e.g., at an angle of approximately 70 degrees to an angle of approximately 110 degrees). However, the disclosure is not limited to such an arrangement.

Distal tip 120 may also include an elevator 126 for changing an orientation of an accessory device or a tool inserted in a working channel of medical device 110. Elevator 126 may alternatively be referred to as a swing stand, pivot stand, raising base, or any suitable other term. Elevator 126 may be pivotable via, e.g., an actuation wire or another control element that extends from handle 112, through shaft 118, to elevator 126.

A distal portion of shaft 118 that is connected to distal tip 120 may have a steerable section 128. Shaft 118 may include a variety of structures that are known or may become known in the art. Described in further detail below, steerable section 128 may comprise an articulation joint assembly having at least six degrees of freedom. A flexible sheath or cover 129 may encompass, or surround, the articulation joint assembly of steerable section 128. The articulation joint assembly comprising steerable section 128 is depicted in the following figures with cover 129 removed.

Handle 112 may have one or more actuators/control mechanisms 130. One or more of control mechanisms 130 may provide control over steerable section 128. One or more of control mechanisms 130 may allow for provision of air, water, suction, etc. For example, handle 112 may include control knobs 132, 134 for left, right, up, and/or down control of steerable section 128. For example, one of knobs 132, 134 may provide left/right control of steerable section 128, and the other of knobs 132, 134 may provide up/down control of steerable section 128. Although not shown in FIG. 1, handle 112 may include additional actuators/control mechanisms 130. The additional actuators/control mechanisms 130 may be configured to control additional aspects of steerable section 128. For example, as described in further detail below, the additional actuators/control mechanisms 130 may be configured to extend, retract, slide, or twist steerable section 128.

Although control knobs 132, 134 are depicted in FIG. 1, it will be appreciated that alternative actuator types may be used to control steerable section 128. For example, a handle 112 may include a joystick that is movable in left/right and up/down directions. The joystick may also be pushed distally or pulled proximally to move distal tip 120 distally or proximally, respectively. Twisting of the joystick may cause rotation of steerable section 128 about a central longitudinal axis of steerable section 128. Laterally shifting the joystick may cause distal tip 120 to move laterally (left/right/up (forward)/down (backward).

A plurality of actuation elements, such as cables or wires suitable for medical procedures (e.g., medical grade plastic or metal), may extend from handle 112, through shaft 118 to steerable section 128. In some examples, at least some of the cables or wires may extend to distal tip 120. Shown more clearly in the following figures, each actuation element may extend at least partially through a Bowden sheath. For example, actuation elements 154 and Bowden sheaths 156 are shown between adjacent links of steerable section 128 in the following figures.

Handle 112 may further include one or more locking mechanisms 136 (e.g., knobs or levers) for preventing steering and/or braking of steerable section 128 in at least one of an up, down, left, or right direction. Handle 112 may include an elevator control lever 138. Elevator control lever 138 may raise and/or lower elevator 126, via a connection between lever 138 and an elevator control element. The elevator control element configured to raise and/or lower elevator 126 may extend from lever 138, through shaft 118 and steerable section 128, and to elevator 126. A port 140 may allow passage of a tool through port 140, into a working channel of the medical device 110, to distal tip 120.

In use, an operator may insert at least a portion of shaft 118 into a body lumen of a subject. Distal tip 120 may be navigated to a procedure site in the body lumen. For example, the operator may push or urge distal tip 120 distally in order to advance distal tip 120 through the body lumen. The operator may insert an accessory device (not shown) into port 140, and pass the accessory device through shaft 118 via a working channel to distal tip 120. The accessory device may exit the working channel at distal tip 120. The user may use elevator control lever 138 to raise elevator 126 and angle the accessory device toward a desired location (e.g., a papilla of the pancreatico-biliary tract). The user may use the accessory device to perform a medical procedure. The user may rotate one or more actuators (e.g., control knobs 132, 134, or a joystick, etc.), to bend, or articulate, steerable section 128 in one or more directions and/or to extend or retract steerable section 128.

The configuration of handle 112 shown in FIG. 1 is merely exemplary. For example, in alternative configurations, handle 112 of medical device 110 may be configured as a joystick, as discussed above. The joystick may have any or all of the characteristics of joysticks commonly used in the art. In these aspects, a stick of the joystick may be configured to control a direction of articulation (e.g., up, down, left, or right) of steerable section 128; a trigger may be configured to extend/retract steerable section 128; at least one actuator or button may be configured to actuate elevator 126. Alternative configurations of the joystick are also contemplated.

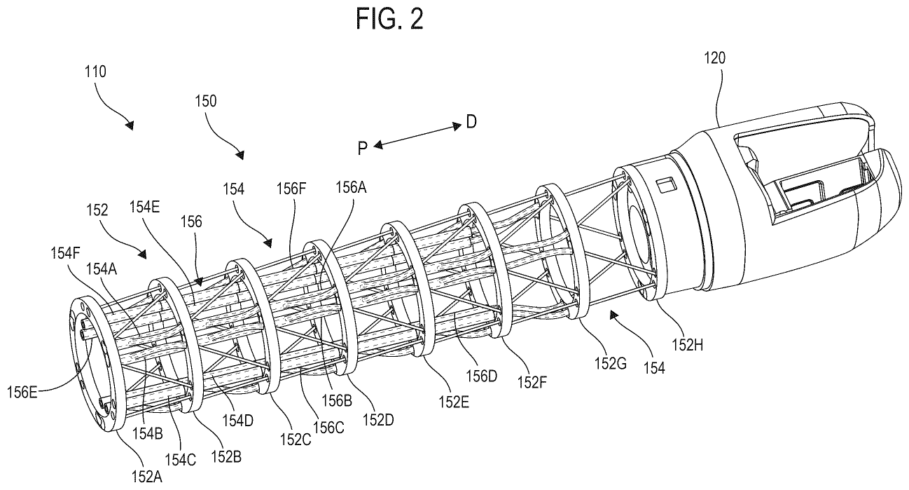

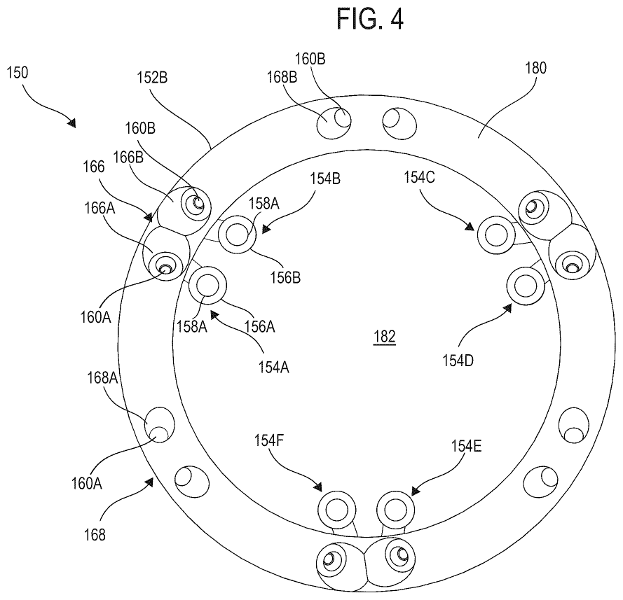

FIG. 2 illustrates an articulation assembly 150 of medical device 110 in a straight configuration, as well as a housing of distal tip 120 (FIG. 2 omits components of distal tip 120 for simplicity of illustration). Articulation assembly 150 may be an element of steerable section 128 of FIG. 1. For example, articulation assembly 150 may be disposed beneath cover 129 of steerable section 128 of FIG. 1. Articulation assembly 150 may include a plurality of discs 152 controlled via a plurality of actuation elements 154. Aspects of each disc of the plurality of discs 152 are shown and described in more detail in FIG. 4.

Articulation assembly 150 may further include a plurality of Bowden sheaths 156. Bowden sheath 156 may alternatively be called a cable housing, a tube, or a pipe). Each of actuation elements 154 may extend at least partially through a respective Bowden sheath 156, and may be movable relative to the Bowden sheath 156 that actuation element 154 extends through. In these aspects, portions of each actuation element 154 are shown in broken lines, for example, to illustrate actuation element 154 extending through a respective Bowden sheath 156. Although not shown, a proximal end of each actuation element 154 may be coupled to one or more control mechanisms 130 (FIG. 1) of handle 112. A distal end of each actuation element 154 may be coupled to distal tip 120 and/or a distal portion of articulation assembly 150.

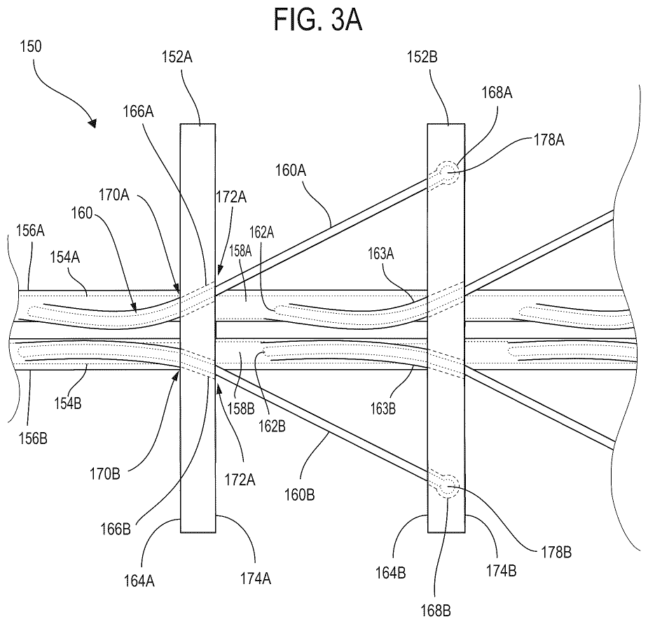

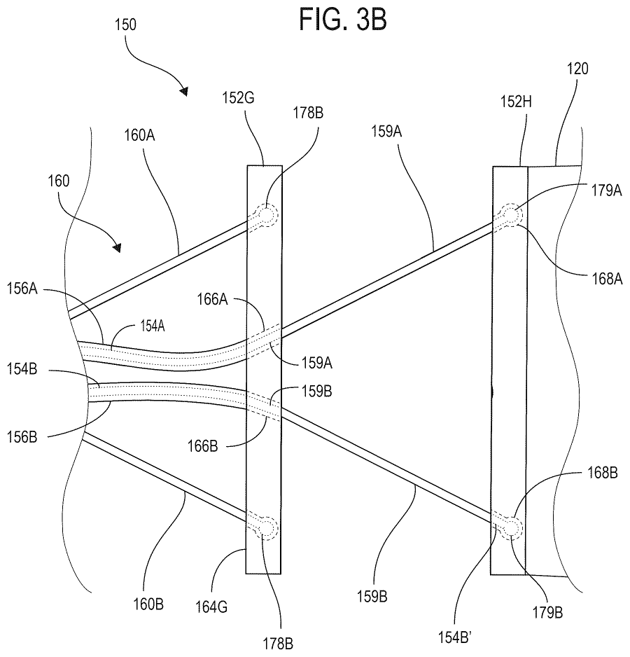

Six actuation elements 154 and six Bowden sheaths 156 are shown in FIG. 2. Specific actuation elements 154 and Bowden sheaths 156 are identified using letters following the element number in FIG. 2 and subsequent figures, where appropriate. For example, medical device 110, including articulation assembly 150, may include: a first actuation element 154A extending at least partially through a first Bowden sheath 156A, a second actuation element 154B extending at least partially through a second Bowden sheath 156B, a third actuation element 154C extending at least partially through a third Bowden sheath 156C, a fourth actuation element 154D extending at least partially through a fourth Bowden sheath 156D, a fifth actuation element 154E extending at least partially through a fifth Bowden sheath 156E, and a sixth actuation element 154F extending at least partially through a sixth Bowden sheath 156F. Aspects of actuation elements 154 and Bowden sheaths 156 are shown and described in more detail with respect to FIGS. 3A and 3B.

The plurality of discs 152 may be dispersed evenly from one another in the straight configuration. Each of discs 152 may have an annular, washer shape. A longitudinal axis may extend through a center of a central opening of each disc 152 in the straight configuration of articulation assembly 150. Eight discs 152 are shown in FIG. 2 and FIGS. 5A-8D, with individual discs 152 identified using letters following the element number. For example, articulation assembly 150 may include a first, proximalmost disc 152A, a second disc 152B, a third disc 152C, a fourth disc 152D, a fifth disc 152E, a sixth disc 152F, a seventh disc 152G, and an eighth, distalmost disc 152H. Eighth disc 152H may be coupled to a proximal end of distal tip 120. Although eight discs 152 are shown, fewer or more discs 152 may be utilized with articulation assembly 150.

In the straight configuration, each disc 152 may be separated from one another by a defined distance. The distance between each disc 152 may be the same along an entire length of articulation assembly 150. Alternatively, the distance between each disc may be different along the entire length of articulation assembly 150. In other words, some discs 152 may be closer to one another as compared to others. For example, each disc 152 of articulation assembly may be unconnected from an adjacent disc 152. Furthermore, each disc 152 may not contact an adjacent disc 152. In these aspects, actuation elements 154, including arms 160, may be configured to control the location of discs 152 relative to one another (e.g., without being connected or contacting adjacent discs 152).

A proximal portion of articulation assembly 150 is shown in FIG. 3A. A distal portion of articulation assembly 150 is shown in FIG. 3B. For ease of description, portions of only two actuation elements (e.g., first actuation element 154A and second actuation element 154B) and their respective Bowden sheaths (e.g., first Bowden sheath 156A and second Bowden sheath 156B) are shown in FIGS. 3A and 3B. Additionally, first, proximalmost disc 152A and second disc 152B is shown in FIG. 3A. Although not shown, first disc 152A may be fixed to a distal end of shaft 118 (FIG. 1). Seventh disc 152G, and eighth, distalmost disc 152H are shown in FIG. 3B. Eighth disc 152H may be fixed to a proximal end of distal tip 120. Aspects of actuation elements 154A, 154B are shown in broken lines in the following figures, for example, to illustrate actuation elements 154A, 154B extending through their respective Bowden sheaths 156A, 156B and through portions of discs 152A, 152B, 152G, 152H. Similarly, aspects of discs 152A, 152B, 152G, 152H are shown in broken lines, for example, to illustrate internal portions of discs 152A, 152B, 152G, 152H.

Referring primarily to FIG. 3A, each actuation element 154A, 154B may have a primary stem 158A, 158B and a plurality of arms 160A, 160B extending radially outward from their respective primary stem 158A, 158B. Each branch of the plurality of arms 160A, 160B may extend from respective primary stem 158A, 158B via a joint, or a fork 162A, 162B. Each branch of the plurality of arms 160A, 160B may extend distally relative to fork 162A, 162B. The plurality of arms 160A, 160B may be integrally formed with primary stem 158A, 158B, respectively. In other aspects, the plurality of arms 160A, 160B may be separate portions coupled to primary stem 158A, 158B. Each arm 160A, 160B may have a diameter that is smaller than a diameter of primary stem 158A, 158B.

In examples, each actuation element 154A, 154B may be a cable comprising a plurality of wires (e.g., a plurality of twisted wires). A wire of the plurality of wires may form each of arms 160. A proximal portion of each actuation element 154A, 154B may include all of the wires of the plurality of wires. The plurality of wires that are still wound (or otherwise held) together may comprise stem 158A, 158B. Wires of the plurality of wires may branch off of their respective stems 158A, 158B at successive locations (forks 162A, 162B) moving distally along actuation elements 154A, 154B. Thus, more distal portions of stems 158A, 158B may comprise fewer wires than more proximal portions of stems 158A, 158B. In some examples, Bowden sheaths 156 may be omitted. In such examples, one or more wires of actuation element 154A, 154B may be coupled to distalmost disc 152H.

A specific number of arms 160A, 160B extending from primary stem 158A, 158B of each actuation element 154A, 154B may be related to a total number of discs 152 comprising articulation assembly 150. For example, each actuation element of an articulation assembly having eight discs 152 may have at least seven arms 160 extending from each respective primary stem 158. Each actuation element 154 of an articulation assembly 150 having seven discs 152 may have at least six arms 160 extending from each respective primary stem 158. In alternatives, a number of arms 160 may be equal to a number of discs 152.

As previously described, primary stem 158A, 158B of each actuation element 154A, 154B may extend at least partially through Bowden sheath 156A, 156B, respectively. In some aspects, Bowden sheath 156A, 156B may be a primary Bowden cable. In such aspects, a portion of each arm 160A, 160B (e.g., a proximal portion of arm 160A, 160B) of actuation elements 154A, 154B may extend at least partially through a secondary Bowden sheath 163A, 163B, for example, sized and shaped to receive arm 160A, 160B. In these aspects, secondary Bowden sheath 163A, 163B may have a diameter that is smaller than a diameter of primary Bowden sheath 156A, 156B.

Secondary Bowden sheath 163A, 163B may be integrally formed with primary Bowden sheath 156A, 156B, respectively. In other aspects, secondary Bowden sheath 163A, 163B may be a separate component fixed or joined to primary Bowden sheath 156A, 156B. Secondary Bowden sheath 163A, 163B may extend between fork 162A, 162B of arm 160A, 160B and a proximal face of a distal adjacent disc 152. For example, secondary Bowden sheath 163A, 163B may extend between fork 162A, 162B of a proximalmost arm 160A, 160B to a proximal face 164A of first disc 152A. Another secondary Bowden sheath 163A, 163B may extend between a second-most proximal fork 162A, 162B of arm 160A, 160B and a proximal face 164B of adjacent disc 152B. In some aspects, secondary Bowden sheath 163A, 163B may extend at least partially and distally through the adjacent disc 152 (e.g., second disc 152B).

Each arm 160A, 160B may extend at least partially through at least two adjacent discs 152 that are distal to fork 162A, 162B. Each arm 160A, 160B may extend first through a channel 166A, 166B of a disc 152 (e.g., first disc 152A) that is immediately adjacent to fork 162A, 162B, and then through/into a blind hole 168A, 168B of a next, distal disc 152 (e.g., second disc 152B).

A proximal opening 170A, 170B of each respective channel 166A, 166B may be disposed on proximal face 164A, 164B of each disc 152A, 152B. A distal opening 172A, 172B of each respective channel 166A, 166B may be disposed on a distal face 174A, 174B of each disc 152A, 152B. As each channel 166A, 166B extends distally, each channel 166A, 166B may be angled away from the longitudinal axis of articulation joint 150. Each blind hole 168A, 168B may be angled away from the longitudinal axis of articulation joint 150. For example, each channel 166A, 166B may have a non-zero angle with respect to the central longitudinal axis of articulation assembly 150. Similarly, each blind hole 168A, 168B may have a non-zero angle with respect to the central longitudinal axis of articulation assembly 150.

Each arm 160A, 160B may extend distally relative to distal opening 172A, 172B and a least partially through a disc 152 (e.g., second disc 152B) that is adjacent to a disc through which arm 160A, 160B fully extended (e.g., first disc 152A). For example, a distal end of each arm 160A, 160B may extend into a blind hole 168A, 168B of second disc 152B. Each arm 160A, 160B may be angled across a space 176 defined between adjacent discs 152 (may not be parallel to a central longitudinal axis of articulation assembly 150).

A distal end of each arm 160A, 160B may include a termination feature 178A, 178B. Termination feature 178A, 178B may be a widened or protruding portion (e.g., a ferrule, crimp, or integrally formed protruding portion). A diameter of each termination feature 178A, 178B may be greater than a diameter of each arm 160A, 160B. In some aspects, termination feature 178A, 178B may be coupled to or integrally formed with the distal end of each arm 160A, 160B. Each termination feature 178A, 178B may be configured to secure the distal end of each arm 160A, 160B to disc 152B. Similarly, blind holes 168A, 168B may be sized and shaped to secure termination feature 178A, 178B to second disc 152B.

The configuration of actuation elements 154A, 154B described above may be repeated for each of arms 160A, 160B between second disc 152B and sixth disc 152F (e.g., a third-to-last disc). The configuration of actuation elements 154A, 154B through the remaining two discs 152 (e.g., seventh disc 152G and eighth disc 152H) is described below.

A distal portion 159A, 159B of primary stem 158A, 158B of each actuation elements 154A, 154B may extend through channel 166A, 166B of the second-to-last disc (e.g., seventh disc 152G). In these aspects, a distal end of primary Bowden sheath 156A, 156B may abut against proximal face 164G of seventh disc 152G. A distal end of each distal portion 159A, 159B may include a termination feature 179A, 179B. In these aspects, termination feature 179A, 179B of distal portion 159A, 159B may be secured within blind holes 168A, 168B of distalmost disc 152H.

FIG. 4 illustrates a cross-section of articulation assembly 150, including a cross-section of second disc 152B, for example, taken at a transverse plane (e.g., a plane that is perpendicular to the longitudinal axis of articulation assembly 150). A distal end of second disc 152B may extend into the page. Each disc of the plurality of discs 152 may have any or all of the same characteristics described below with respect to second disc 152B. However, in some aspects, a proximalmost disc (disc 152A) and a distalmost disc (disc 152H) may include fewer openings/holes than those described below.

For simplicity, only a single pair of channels 166, including a first channel 166A and a second channel 166B, is identified in FIG. 4. Similarly, only a single pair of blind holes 168 are identified in FIG. 4. Only a first blind hole 168A and a second blind hole 168B are identified in FIG. 4. Each actuation element 154 (e.g., first actuation element 154A, second actuation element 154B, third actuation element 154C, fourth actuation element 154D, fifth actuation element 154E, and sixth actuation element 154F) are identified in FIG. 4. However, for simplicity, particular aspects of only first actuation element 154A and second actuation element 154B are identified in further detail in FIG. 4. The remaining actuation elements (e.g., third actuation element 154C, fourth actuation element 154D, fifth actuation element 154E, sixth actuation element 154F) may have any or all of the characteristics of first actuation element 154A and second actuation element 154B.

Disc 152B may include an annular body 180 defining a central opening 182. Primary stem 158A, 158B of each actuation element 154A, 154B may extend through central opening 182. Similarly, primary Bowden sheath 156A, 156B of each actuation element 154A, 154B may extend through central opening 182.

Although not shown, additional wires, cables, tubes, components, etc. may extend through central opening 182. Channels 166 and blind holes 168 may be arranged around central opening 182 of body 180 in pairs and in an alternating pattern. The pairs of channels 166 and blind holes 168 may be evenly or unevenly spaced about central opening 182. Each pair of channels 166 may be circumferentially offset from each pair of blind holes 168.

Channels 166 and blind holes 168 may be angled relative to one another. For example, the channels of each pair of channels 166 may be angled away from one another as each channel extends in a distal direction. Conversely, the blind holes of each pair of blind holes 168 may be angled towards one another as each blind hole extends in the distal direction. Respective arms 160A, 160B of actuation elements 154A, 154B may extend distally through channels 166 and blind holes 168, as described above with respect to FIG. 3A. The angled channels 166 and blind holes 168 may, for example, prevent kinks or bends of each arm 160A, 160B extending through a respective channel 166 and blind hole 168.

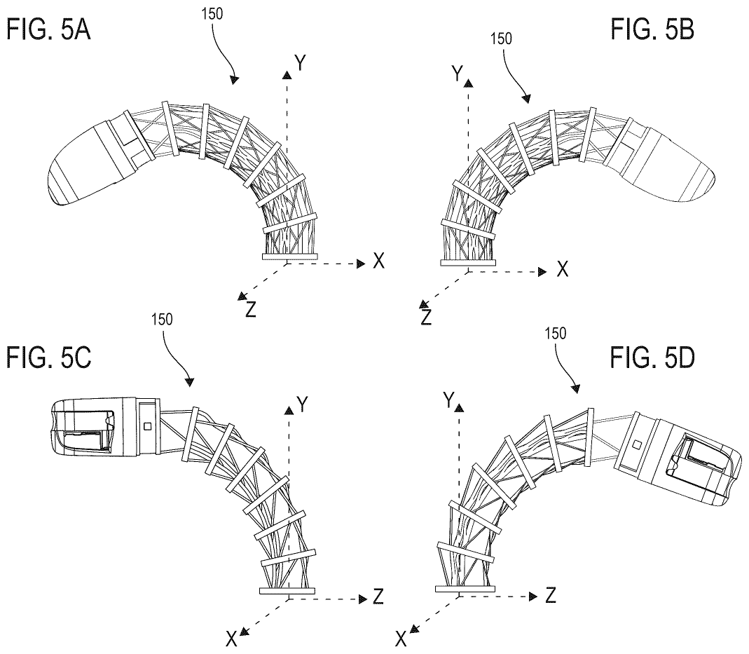

FIGS. 5A-9D illustrate exemplary configurations of articulation assembly 150.

FIGS. 5A, 5B, 5C, and 5D illustrate articulation assembly 150 bent in four directions (in two degrees of freedom). Each of the four directions may be 90 degrees apart from each other about a longitudinal axis of articulation assembly 150 (e.g., up, down, left and right directions). For example, FIG. 5A illustrates articulation assembly 150 bent in a first direction (e.g., up) which may be achieved by pulling proximally, or shortening, actuation element 154A, 154E (thus pulling corresponding arms 160A, 160E proximally) while pushing distally, or lengthening, actuation elements 154C, 154D (thus pushing distally, or lengthening corresponding arms 160C, 160D).

FIG. 5B illustrates articulation assembly 150 bent in a second direction (e.g., down) which may be achieved by pulling actuation elements 154C, 154D proximally (thus pulling corresponding arms 160C, 160D proximally) while pushing actuation elements 154A, 154E distally (thus pushing arms 160A 160E distally).

FIG. 5C illustrates articulation assembly 150 bent in a third direction (e.g., left) which may be achieved by pulling actuation element 154E proximally (thus pulling arm 160E proximally) while pushing actuation element 154B distally (thus pushing arm 160B distally).

FIG. 5C illustrates articulation assembly 150 in a fourth direction (e.g., right) which may be achieved by pulling actuation element 154B proximally (thus pulling arm 160B proximally) while pushing actuation element 154E distally (thus pushing arm 160E distally). For example, FIG. 2 illustrates a first, straight configuration of articulation assembly 150. FIGS. 5A-5D illustrate a second, a third, a fourth, and a fifth configuration, respectively, of articulation assembly 150. Articulation assembly 150 may transition from the first configuration to any of the second, third, fourth, or fifth configurations, and vice versa.

Such configurations may be achieved by pulling one or more actuation elements proximally and/or pushing one or more actuation elements distally. For example, one of a maximum bend angle of articulation joint 150 in each of the four directions may be equivalent. In other words, articulation joint 150 may bend in each of the four directions equally.

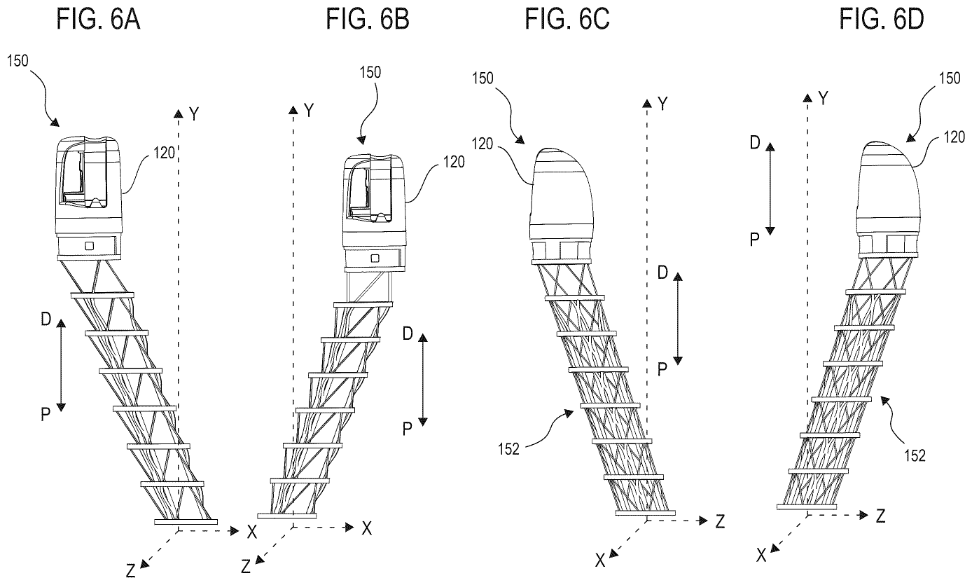

FIGS. 6A-6D illustrate articulation assembly 150 in four sliding configurations. For example, FIG. 6A illustrates articulation assembly 150 slid in a first direction (e.g., a −X direction) which may be achieved by pushing actuation elements 154B, 154E distally (thus pushing arms 160B, 160E distally).

FIG. 6B illustrates articulation assembly 150 slid in a second direction (e.g., a +X direction) which may be achieved by pulling actuation elements 154B, 154E proximally (thus pulling arms 160B, 160E proximally).

FIG. 6C illustrates assembly 150 slid in a third direction (e.g., a +Z direction) which may be achieved by pulling actuation elements 154F, 154D proximally (thus pulling arms 160F, 160D proximally) while pushing actuation element 154A distally (thus pushing arms 160A, 160C distally).

FIG. 6D illustrates assembly 150 slid in a fourth direction (e.g., a -Z direction) which may be achieved by pulling actuation elements 154A, 154C proximally (thus pulling arms 160A, 160C proximally) while pushing actuation elements 154D, 154F distally (thus pushing arms 160D, 160F distally. In these aspects, FIGS. 6A-6D illustrate a sixth, a seventh, an eighth, and a ninth configuration, respectively, of articulation assembly 150. Articulation assembly 150 may transition from any of the previous configurations to any of the sixth, seventh, eighth, or ninth configurations, and vice versa.

In the sliding configurations of articulation assembly 150, each disc 152 may be configured to slide (translate laterally) relative to one another. In such a configuration, each successively distal disc 152 may be more offset from the central longitudinal axis of articulation assembly 150. For example, a distance between the longitudinal axis and a distalmost disc 152 may be greater, as compared to a distance between the longitudinal axis and a proximalmost disc 152. A longitudinal distance between each disc 152 may be maintained (e.g., a distance between a central point of each disc 152 measured about the Y-axis). In each of the four sliding configurations, an orientation of distal tip 120 may be maintained. For example, a longitudinal axis of distal tip 120 may be parallel to the Y-axis.

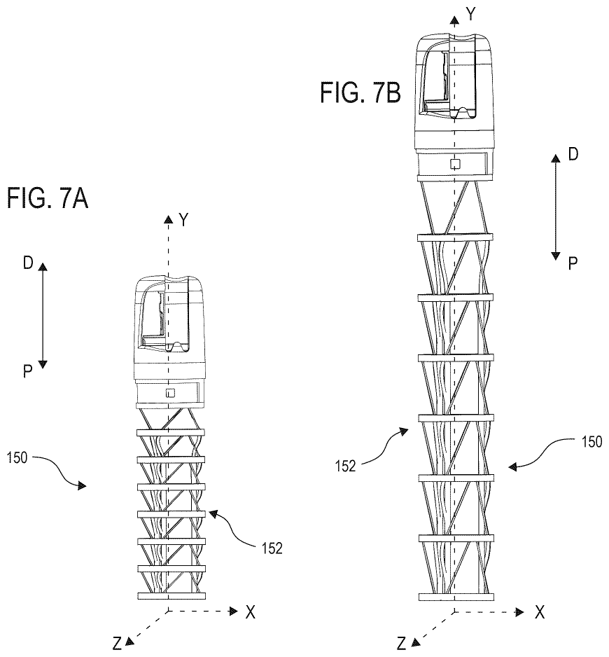

FIGS. 7A and 7B illustrate articulation assembly 150 in a retracted configuration (FIG. 7A) and in an extended configuration (FIG. 7B). In the retracted configuration (FIG. 7A) which may be achieved by pulling all actuation elements 154 proximally at a same time, each disc 152 of articulation assembly 150 may be spaced by a smaller distance (e.g., as compared to the straight configuration shown in FIG. 2 or the extended configuration shown in FIG. 7B). In the extended configuration (FIG. 7B), which may be achieved by extending all actuation elements 154 distally at a same time, each disc 152 of articulation assembly 150 may be spaced by a greater distance (e.g., as compared to the straight configuration shown in FIG. 2 or the retracted configuration shown in FIG. 6A). In these aspects, an overall length of articulation assembly 150 may be increased, as shown in FIG. 7A, or decreased, as shown in FIG. 7B. For example, FIG. 2 illustrates a nominal configuration of articulation assembly 150 having a first length.

FIGS. 7A and 7B illustrate a tenth and an eleventh configuration of articulation assembly 150, respectively. In these aspects an overall length of articulation assembly 150 in the tenth configuration (FIG. 7A) may be less than the overall length of articulation assembly 150 in the first configuration (FIG. 2). Conversely, an overall length of articulation assembly 150 in the eleventh configuration (FIG. 7B) may be greater than the overall length of articulation assembly 150 in the first configuration (FIG. 2) and the overall length of articulation assembly 150 in the tenth configuration (FIG. 7A). Articulation assembly 150 may transition from any configuration previously described, to the tenth or eleventh configurations, and vice versa.

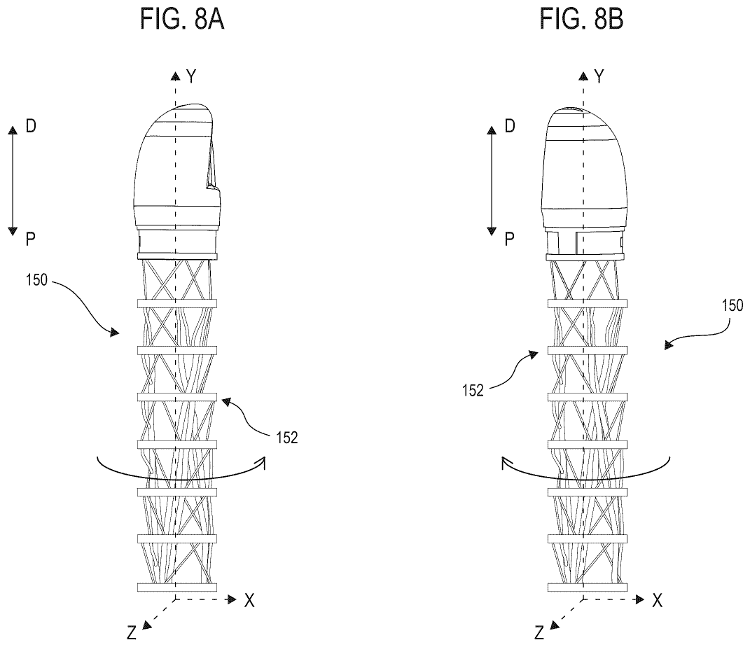

FIGS. 8A and 8B each illustrate articulation assembly 150 in a first twisted configuration (FIG. 8A) and in a second twisted configuration (FIG. 8B). For example, articulation assembly 150 may be configured to twist clockwise (FIG. 8A), which may be achieved by pulling actuation elements 154B, 154D, 154F proximally, while pushing actuation elements 154A, 154C, 154E distally. Additionally or alternatively, articulation assembly 150 may be configured to twist counterclockwise (FIG. 8B) which may be achieved by pulling actuation elements 154A, 154C, 154C proximally while pushing actuation elements 154B, 154D, and 154F distally. In each twisted configuration, each disc 152 may be twisted relative to one another in a clockwise or counterclockwise direction. For example, articulation assembly 150 may be configured to twist in at least one direction about a central longitudinal axis of the actuation assembly. FIGS. 8A and 8B illustrate a twelfth and a thirteenth configuration, respectively, of articulation assembly 150. Articulation assembly 150 may transition from any of the configurations previously described to the twelfth or thirteenth configuration, and vice versa.

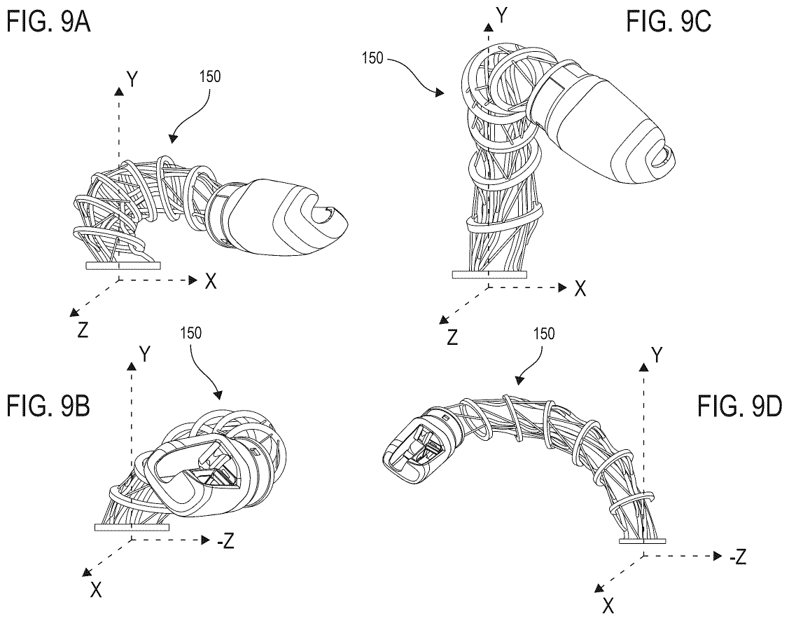

FIGS. 9A-9D illustrate different views of an exemplary configuration of articulation assembly 150 that include movement in multiple degrees of freedom. For example, articulation assembly 150 may be configured to bend, twist, slide and extend/contract as described above with respect to FIGS. 4A-8B. For example, articulation assembly 150 may be configured to bend in the -Z direction and in the X direction. Additionally, articulation assembly 150 may be configured to twist and/or retract, as described above. FIGS. 9A and 9B illustrate a fourteenth and fifteenth configuration, respectively, of articulation assembly 150. Articulation assembly 150 may transition from any of the configurations previously described to the fourteenth or fifteenth configurations, and vice versa. For example, articulation assembly may be configured to bend in the Z-and X-directions. Additionally, articulation assembly 150 may be configured to twist and/or extend, as described above. FIGS. 9C and 9D illustrate a sixteenth and seventeenth configuration, respectively, of articulation assembly 150. Articulation assembly 150 may transition from any of the configurations previously described to the sixteenth or seventeenth configurations, and vice versa.

The exemplary configurations of articulation assembly 150 shown in FIGS. 5A-9D may be achieved during use. For example, a user may bend articulation assembly 150 in any of the configurations shown in FIGS. 5A-9D to acquire various views of a subject's lumen.

In some aspects, each actuation element 154 of the plurality of actuation elements may have a variable length. For example, each actuation element 154 may be configured to extend or retract along its longitudinal axis, as described above. The variable length of each actuation element 154 may allow for additional degrees of freedom in the movement and articulation of assembly 150. In some cases, the extension or retraction of one or more actuation elements 154 may be controlled independently of the others. This variable length capability may enable a greater range of positions and orientations of distal tip 120. The ability to extend or retract individual actuation elements 154 may also contribute to the various configurations of articulation assembly 150 described above, including bending, sliding, twisting, and extending/retracting movements.

In other examples, at least one of the actuation elements 154 may have a fixed length. In such cases, articulation assembly 150 may bend in one plane so long as the actuation element with the fixed length is not changing in length. As a result of one or more actuation elements 154 having a fixed length, different configurations of articulation assembly 150 may be achieved. For instance, the articulation assembly 150 may be configured to bend in fewer directions or planes compared to configurations where all actuation elements 154 have variable lengths. In some aspects, the fixed-length actuation element 154 may act as a constraint or pivot point around which the other variable-length actuation elements 154 can manipulate articulation assembly 150. This arrangement may allow for more controlled or limited articulation in certain applications where full range of motion is not required or desired. The combination of fixed-length and variable-length actuation elements 154 may provide a balance between flexibility and stability in the articulation assembly 150.

It will be apparent to those skilled in the art that various modifications and variations can be made to the disclosed device without departing from the scope of the disclosure. For examples, the configuration of gaps and links and the bend angles may be altered to suit any medical device. It will be understood that the bend angles, sizes of each gap, and/or the number of gaps and links are not limited to the examples described herein. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An articulation assembly for a medical device, the articulation assembly including:
a plurality of discs, and
a plurality of actuation elements, wherein each actuation element includes a primary stem and a plurality of arms extending radially outward from the primary stem, and wherein each arm of the plurality of arms extends at least partially through at least two adjacent discs of the plurality of discs.

2. The articulation assembly of claim 1, wherein each disc of the plurality of discs is equally spaced from one another in a straight configuration.

3. The articulation assembly of claim 1, wherein each arm of the plurality of arms is configured to extend distally through a respective channel of a first disc of the at least two adjacent discs.

4. The articulation assembly of claim 3, wherein each respective channel has a non-zero angle with respect to a central longitudinal axis of the articulation assembly.

5. The articulation assembly of claim 3, wherein a distal end of each arm includes a termination feature, and wherein the termination feature is secured within a second, distal disc of the at least two adjacent discs.

6. The articulation assembly of claim 1, wherein the articulation assembly is configured to bend in at least four directions.

7. The articulation assembly of claim 1, wherein, in a first configuration of the articulation assembly, the articulation assembly has a first length, and wherein, in a second configuration of the articulation assembly, the articulation assembly has a second length, wherein the second length is greater than the first length.

8. The articulation assembly of claim 7, wherein, in a third configuration of the articulation assembly, the articulation assembly has a third length, wherein the third length is less than the first length and the second length.

9. The articulation assembly of claim 1, wherein the plurality of discs is configured to slide laterally relative to one another.

10. The articulation assembly of claim 1, wherein each arm of the plurality of arms is coupled to the primary stem at a fork.

11. The articulation assembly of claim 10, wherein the primary stem of each actuation element extends at least partially through a respective Bowden sheath.

12. The articulation assembly of claim 11, wherein the Bowden sheath is a primary Bowden sheath, and wherein a portion of each arm of the plurality of arms extends through a secondary Bowden sheath, wherein the secondary Bowden sheath extends between the joint and a proximal face of a first, proximal disc of the at least two adjacent discs.

13. The articulation assembly of claim 1, wherein each disc of the plurality of discs includes a body defining a central opening, wherein the primary stem of each actuation elements of the plurality of actuation elements extends through the central opening.

14. The articulation assembly of claim 1, wherein the plurality of discs includes at least six discs, and wherein the plurality of actuation elements includes at least six actuation elements.

15. The articulation assembly of claim 1, wherein the articulation assembly is configured to twist in at least one direction about a central longitudinal axis of the articulation assembly.

16. An articulation assembly for a medical device, comprising:
a plurality of discs, wherein each disc includes a body defining a central opening; and
a plurality of actuation elements arranged in pairs, wherein each actuation element includes a primary stem and a plurality of arms extending radially outward from the primary stem, and wherein a distal end of each arm is fixedly coupled within the body of a disc of the plurality of discs.

17. The articulation assembly of claim 16, wherein each arm of the plurality of arms is configured to extend distally through a respective channel of the body of at least one disc of the plurality of discs, and wherein the respective channel has a non-zero angle with respect to a central longitudinal axis of the articulation assembly.

18. The articulation assembly of claim 16, wherein each disc of the plurality of discs are configured to slide laterally relative to one another in at least one configuration.

19. An articulation joint for a medical device, comprising:
at least six discs, wherein each disc of the at least six discs includes a body defining a central opening; and
at least six actuation elements wherein each actuation element includes a primary stem and a plurality of arms extending radially outward from the primary stem, and wherein a distal end of each arm of the plurality of arms is fixedly coupled within each of the at least six discs.

20. The articulation joint of claim 19, wherein each arm of the plurality of arms is configured to extend distally through a respective channel of each disc of the at least six discs.
